(19) United States
(12) Patent Application Publication
(10) Pub. No.: US 2023/0032068 A1
(43) Pub. Date: Feb. 2, 2023

(54) SYSTEM AND METHOD FOR ESTIMATING A RESERVOIR VOLUME OF AN IMPLANTABLE MEDICAL DEVICE

(71) Applicant: Medtronic, Inc., Minneapolis, MN (US)

(72) Inventors: Yan Liu, Maple Grove, MN (US); Scott A. Hareland, Lino Lakes, MN (US)

(21) Appl. No.: 17/816,001

(22) Filed: Jul. 29, 2022

Related U.S. Application Data
(60) Provisional application No. 63/228,425, filed on Aug. 2, 2021.

(51) Int. Cl. A61M 5/142

(52) U.S. Cl. CPC A61M 5/14276; A61M 2205/3523; A61M 2205/18

(57) ABSTRACT

A medical system configured to estimate a volume of infusate within an implantable medical device, the system including an implantable medical device comprising a fluid reservoir, and an external programmer in communication with the implantable medical device, the external programmer comprising a processor configured to estimate a distribution of refill intervals based on actual infusion data including patient initiated infusions.

### CROSS-REFERENCE TO RELATED APPLICATION

The present application claims priority to U.S. Provisional Application Ser. No. 63/228,425, filed Aug. 2, 2021, the entirety of which is incorporated herein by reference.

### TECHNICAL FIELD

The present technology is generally related to implantable medical devices, and more particularly to implantable medical pumps for managing the delivery and dispensation of prescribed therapeutic agents.

### BACKGROUND

Implantable medical devices, such as implantable medical pumps, are useful in managing the delivery and dispensation of prescribed therapeutic agents, nutrients, drugs, infusates such as antibiotics, blood clotting agents, analgesics and other fluid or fluid like substances (collectively “infusate” or “infusates”) to patients in volume- and time-controlled doses as well as through boluses. Such implantable pumps are particularly useful for treating diseases and disorders that require regular or chronic (i.e., long-term) pharmacological intervention, including tremor, spasticity, multiple sclerosis, Alzheimer's disease, Parkinson's disease, amyotrophic lateral sclerosis (ALS), Huntington's disease, cancer, epilepsy, chronic pain, urinary or fecal incontinence, sexual dysfunction, obesity, and gastroparesis, to name just a few. Depending upon their specific designs and intended uses, implantable pumps are well adapted to administer infusates to specific areas within the vasculatures and central nervous system, including the subarachnoid, epidural, intrathecal, and intracranial spaces or provide access to those spaces for aspiration.

Providing access to the cerebrospinal fluid for the administration of infusates or aspiration of fluid has a number of important advantages over other forms of infusate administration. For example, oral administration is often not workable because the systematic dose of the substance needed to achieve the therapeutic dose at the target site may be too large for the patient to tolerate without adverse side effects. Also, some substances simply cannot be absorbed in the gut adequately for a therapeutic dose to reach the target site. Moreover, substances that are not lipid soluble may not cross the blood-brain barrier adequately if needed in the brain. In addition, infusion of substances from outside the body requires a transcutaneous catheter or access with a hypodermic needle, which results in other risks such as infection or catheter dislodgment. Further, implantable pumps avoid the problem of patient noncompliance, namely the patient failing to take the prescribed drug or therapy as instructed.

Such implantable pumps are typically implanted at a location within the body of a patient (typically a subcutaneous region in the lower abdomen) and are connected to a catheter configured to deliver infusate to a selected delivery site in the patient. The catheter is generally configured as a flexible tube with a lumen running the length of the catheter to a selected delivery site in the body, such as the intracranial or subarachnoid space.

Implantable medical pumps of this type often include an expandable fluid reservoir, which is accessible for refill or aspiration through an access port. To enable longer-term, uninterrupted operations, the implantable medical pump is typically refilled with a subsequent volume of infusate prior to the entire initial volume of infusate within the fluid reservoir being dispensed. The refill interval (e.g., the length of time between scheduled refill procedures) is typically based upon an estimated reservoir volume over time. Although various methods for estimating a volume of the fluid reservoir currently exist, further improvements in fluid reservoir volume estimation are always desirable. The present disclosure addresses this concern.

### SUMMARY OF THE DISCLOSURE

The techniques of this disclosure generally relate to estimating a volume of infusate within an implantable medical device, including estimating a distribution of possible refill intervals based on actual infusion data, determining a scheduled refill date occurring after a termination date of the majority of the distribution of potential refill intervals, and determining a probability of any deviation between an expected residual volume and an actual residual volume based on control data gathered from a larger sampling of implantable medical devices.

One embodiment of the present disclosure provides a medical system configured to estimate a volume of infusate within an implantable medical device, the system including an implantable medical device comprising a fluid reservoir, and an external programmer in communication with the implantable medical device, the external programmer comprising a processor configured to estimate a distribution of refill intervals based on actual infusion data including patient initiated infusions.

In one embodiment, the distribution of refill intervals is determined by a random probability distribution model. In one embodiment, the random probability distribution model is at least one of a stochastic model, Markov chain or Monte Carlo simulation. In one embodiment, the distribution of refill intervals is used to determine a scheduled refill date based on a probability that the scheduled refill date will occur after a termination date of a majority of the distribution of refill intervals. In one embodiment, at least 95% of the distribution of refill intervals end before the scheduled refill date.

In one embodiment, the system implements one or more controls to inhibit premature exhaustion of infusate, when an estimated fluid reservoir volume decreases below a threshold limit. In one embodiment, the actual infusion data including patient initiated infusions is used to establish an upper control limit. In one embodiment, patient initiated infusions are limited when the actual infusion data including patient initiated infusions at least one of approaches or is greater than or equal to the upper control limit.

In one embodiment, at least one condition having a potential to impact an actual residual volume of infusate delivered by the implantable medical device is considered in the estimation of the distribution of refill intervals. In one embodiment, the at least one condition includes a measured actual residual volume, drug type, off-label usage, infusion rate, concentration, implant duration, manufacturing tolerance, or combination thereof. In one embodiment, the processor is further configured to estimate a probability of an expected residual volume of infusate within the fluid reservoir differing from an actual residual volume of infusate within the fluid reservoir by greater than or equal to a determined percentage, based on an occurrence of at least one condition. In one embodiment, the probability of an expected residual volume differing from an actual residual volume is determined by a Baysian network.

Another embodiment of the present disclosure provides a medical system configured to estimate a volume of infusate within an implantable medical device, the system including an implantable medical device comprising a fluid reservoir, and an external programmer in communication with the implantable medical device, the external programmer comprising a processor configured to estimate a probability of an expected residual volume of infusate within the fluid reservoir differing from an actual residual volume of infusate within the fluid reservoir by greater than or equal to a determined percentage, based on an occurrence of at least one condition.

In one embodiment, the at least one condition includes a measured actual residual volume, drug type, off-label usage, infusion rate, concentration, implant duration, manufacturing tolerance, or combination thereof. In one embodiment, the probability of an expected residual volume differing from an actual residual volume is determined by a Bayesian network. In one embodiment, the probability of an expected residual volume differing from an actual residual volume is determined as a distribution of values, each value representing a possible difference between an expected residual volume and an actual residual volume. In one embodiment, the distribution of values is determined by a random probability distribution model. In one embodiment, the random probability distribution model is at least one of a stochastic model, Markov chain or Monte Carlo simulation.

Another embodiment of the present disclosure provides a method of estimating a volume of infusate within an implantable medical device, the method including collecting actual infusion data including patient initiated infusions from an implantable medical device including a fluid reservoir, and estimating a distribution of refill intervals based on the actual infusion data. In one embodiment, the distribution of refill intervals is determined by a random probability distribution model.

The details of one or more aspects of the disclosure are set forth in the accompanying drawings and the description below. Other features, objects, and advantages of the techniques described in this disclosure will be apparent from the description in the drawings, and from the claims.

### BRIEF DESCRIPTION OF THE DRAWINGS

The disclosure can be more completely understood in consideration of the following detailed description of various embodiments of the disclosure, in connection with the accompanying drawings, in which:

FIG. 1 is a schematic view depicting a medical system configured to estimate a volume of remaining medicament within a fluid reservoir of an implantable medical device, in accordance with an embodiment of the disclosure.

FIGS. 2A-B are cross sectional views depicting an implantable device configured to estimate a volume of remaining medicament within a fluid reservoir, in accordance with an embodiment of the disclosure.

FIG. 3 is a block diagram of an implantable device and programmer configured to estimate a volume of remaining medicament within a fluid reservoir of the implantable device 102, in accordance with an embodiment of the disclosure.

FIG. 4A is a flowchart depicting a model of estimating a refill interval based on actual daily use condition data, in accordance with an embodiment of the disclosure.

FIG. 4B is a graphical depiction of a daily number of patient initiated boluses with an established upper control limit and lower control limit to inhibit premature exhaustion of infusate as a result of dramatic behavioral change prior to the scheduled refill procedure, in accordance with an embodiment of the disclosure.

FIG. 5 depicts a model of control data used to determine conditional probabilities according to Bayes theorem, in accordance with an embodiment of the disclosure.

FIG. 6A depicts a Bayesian network configured to estimate a volume of remaining medicament within a fluid reservoir of an implantable medical device, in accordance with an embodiment of the disclosure.

FIG. 6B depicts an augmented Bayesian network configured to estimate a volume of remaining medicament within a fluid reservoir of an implantable medical device, in accordance with an embodiment of the disclosure.

FIG. 6C depicts an inverted augmented Bayesian network configured to estimate a volume of remaining medicament within a fluid reservoir of an implantable medical device, in accordance with an embodiment of the disclosure.

FIGS. 7-8, depict network models configured to estimate an actual daily volume for a given implantable device based on a larger collection of data from a plurality of other implantable devices, in accordance with an embodiment of the disclosure.

While embodiments of the disclosure are amenable to various modifications and alternative forms, specifics thereof shown by way of example in the drawings will be described in detail. It should be understood, however, that the intention is not to limit the disclosure to the particular embodiments described. On the contrary, the intention is to cover all modifications, equivalents, and alternatives falling within the spirit and scope of the subject matter as defined by the claims.

### DETAILED DESCRIPTION

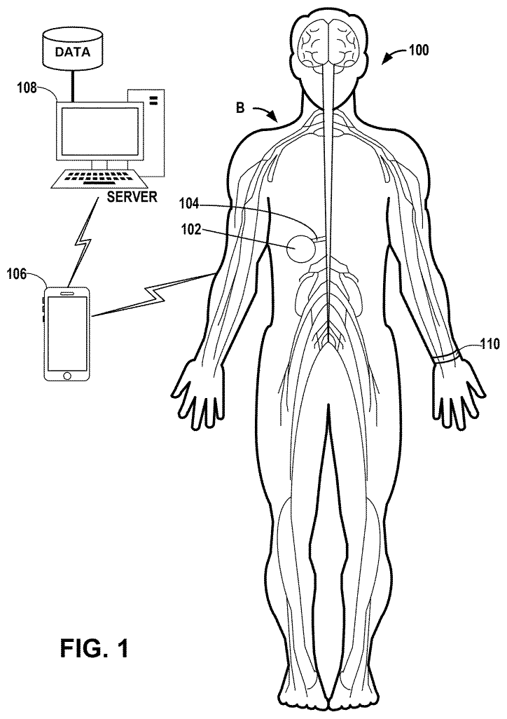

Referring to FIG. 1, a medical system 100 configured to estimate a volume of remaining medicament within a fluid reservoir of an implantable medical device 102, is depicted in accordance with an embodiment of the disclosure. The medical system 100 can include an implantable catheter 104, which in some embodiments can be in fluid communication with the implantable medical device 102, which can be an implantable pump or smart port, configured to dispense infusate over an extended period of time. As depicted, the implantable device 102 can be implanted within the body B of a patient, for example, in an interior torso cavity or in proximity to the patient's ribs or cranially for the introduction of infusate into the patient (e.g., within an intrathecal space, intracranial space, pulmonary artery, etc.) for targeted delivery of infusate. In some embodiments, the implantable device 102 can be placed subcutaneously, and can be held in position by sutures or other retaining features.

Various example embodiments of implantable medical devices, systems and methods are described herein for estimating a volume of remaining medicament within a fluid reservoir of an implantable medical device. Although specific examples of implantable medical pumps are provided, it is to be appreciated that the concepts disclosed herein are extendable to other types of implantable devices. It also to be appreciated that the term “clinician” refers to any individual that can prescribe and/or program a therapeutic regimen with any of the example embodiments described herein or alternative combinations thereof. Similarly, the term “patient” or “subject,” as used herein, is to be understood to refer to an individual or object in which the infusate delivery is to occur, whether human, animal, or inanimate. Various descriptions are made herein, for the sake of convenience, with respect to the procedures being performed by a clinician on a patient or subject (the involved parties collectively referred to as a “user” or “users”) while the disclosure is not limited in this respect.

In some embodiments, the medical system 100 can further include an optional external programmer 106 and optional server 108 configured to communicate with the implantable device 102. In some embodiments, the programmer 106 can be a handheld, wireless portable computing device, such as a cellular telephone, tablet, dedicated implantable device programmer, or the like. Further, in some embodiments, the medical system 100 can include one or more external physiological sensors 110, which can be in communication with the implantable device 102, optional external programmer 106, and optional server 108. In one embodiment, one or more physiological sensors 110 can be incorporated into implantable device 102 or external programmer 106. In one embodiment, a physiological sensor 110 can be worn by the patient (e.g., a smart watch, wristband tracker, sensors embedded in clothing, etc.), carried by the patient (e.g., a smart phone, mobile computing device, etc.), or positioned in proximity to the patient (e.g., a stationary monitor, etc.). Examples of physiological sensors 110 include a heart rate monitor, pulse oximeter, respiratory sensor, perspiration sensor, posture orientation sensor, motion sensor, accelerometer, or the like.

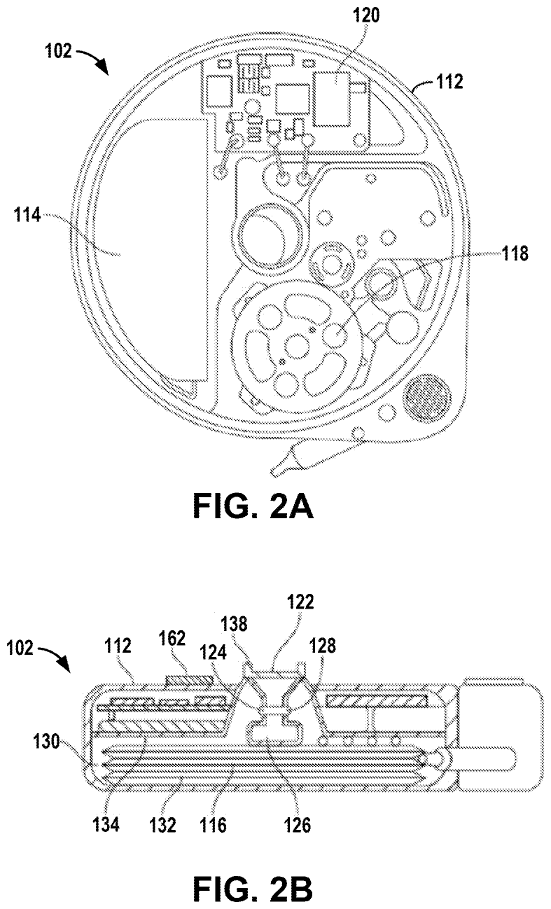

Referring to FIGS. 2A-B, cross sectional views of an implantable device 102 configured to estimate a volume of remaining medicament within a fluid reservoir are depicted in accordance with an embodiment of the disclosure. The implantable device 102 can generally include a housing 112, power source 114, fluid reservoir 116, pump 118, and computing device 120. The housing 112 can be constructed of a material that is biocompatible and hermetically sealed, such as titanium, tantalum, stainless steel, plastic, ceramic, or the like.

The fluid reservoir 116 can be carried by the housing 112 and can be configured to contain infusate. In one embodiment, infusate within the reservoir 116 can be accessed via an access port 122. Accordingly, the access port 122 can be utilized to refill, aspirate, or exchange fluid within the reservoir 116. In some embodiments, the access port 122 can include one or more positional markers 138, for example in the form of a tactile protrusion, one or more lights or LEDs to illuminate through tissue of the patient, an acoustic device to confirm location of the access port 122, and/or one or more wireless location/orientation sensors to aid in positioning of a refilling device relative to the implantable device 102.

In some embodiments, the access port 122 can include a septum 124 configured to seal a port chamber 126 relative to an exterior of the housing 112. The septum 124 can be constructed of a silicone rubber or other material having desirable self-sealing and longevity characteristics. The port chamber 126 can be in fluid communication with the reservoir 116. In one embodiment, the access port 122 can further include an optional needle detection sensor 128, for example in the form of a mechanical switch, resonant circuit, ultrasonic transducer, voltmeter, ammeter, ohmmeter, pressure sensor, flow sensor, capacitive probe, acoustic sensor, and/or optical sensor configured to detect and confirm the presence of an injection needle of a refilling apparatus.

The reservoir 116 can include a flexible diaphragm 130. The flexible diaphragm 130, alternatively referred to as a bellows, can be substantially cylindrical in shape and can include one or more convolutions configured to enable the flexible diaphragm 130 to expand and contract between an extended or full position and an empty position. In one embodiment, the flexible diaphragm 130 can divide the reservoir 116 into an infusate chamber 132 containing liquid infusate (within the flexible diaphragm 130), and a vapor chamber 134 (surrounding the flexible diaphragm 130).

As the infusate chamber 132 is filled with infusate, the flexible diaphragm 130 extends downwardly (with reference to FIG. 2B) toward a bottom portion of the housing 112 until it has reached a maximum volume or some other desired degree of fullness. Alternatively, as the infusate chamber 132 is aspirated, the flexible diaphragm 130 contracts upwardly toward a top portion of the housing 112 until the infusate chamber reaches a minimum volume. In one embodiment, the flexible diaphragm 130 can have a compression spring rate which tends to naturally bias the flexible diaphragm 130 towards an expanded position.

The pump 118 can be carried by the housing 112. The pump 118 can be in fluid communication with the reservoir 116 and can be in electrical communication with the computing device 120. The pump 118 can be any pump sufficient for infusing infusate to the patient, such as a peristaltic pump, piston pump, a pump powered by a stepper motor or rotary motor, a pump powered by an AC motor, a pump powered by a DC motor, electrostatic diaphragm, piezoelectric motor, solenoid, shape memory alloy, or the like.

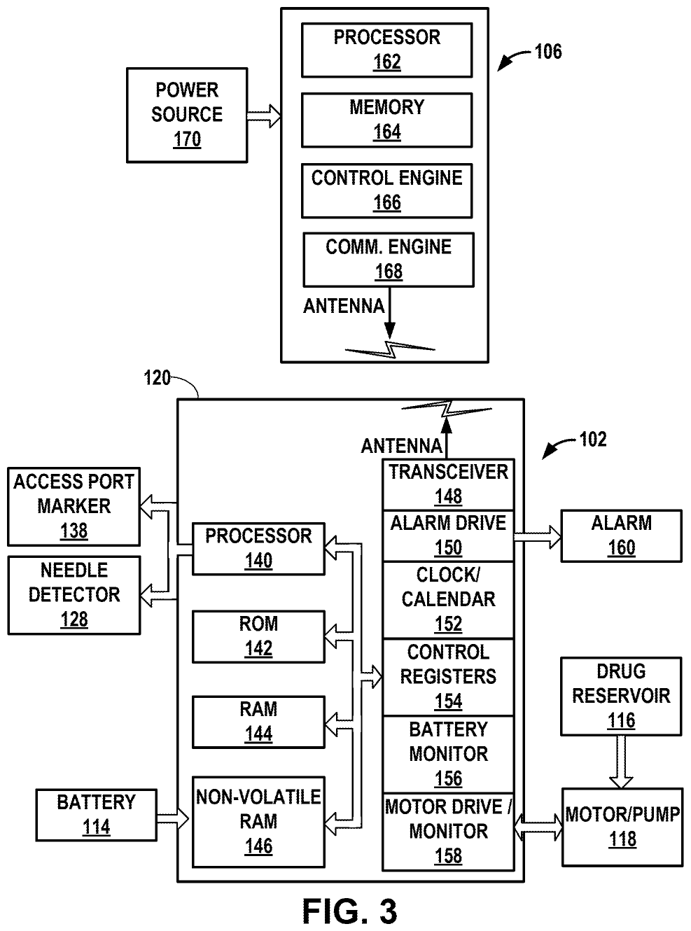

Referring to FIG. 3, a block diagram of an implantable device 102 and programmer 106 configured to estimate a volume of remaining medicament within a fluid reservoir 116 of the implantable device 102, is depicted in accordance with an embodiment of the disclosure. The implantable device 102 can include a computing device 120, which can be carried in the housing 112 (as depicted in FIG. 2A) and can be in electrical communication with the pump 118 and power source 114. The power source 114 can be a battery, such as a rechargeable lithium-ion battery, nickel cadmium battery, or the like. The power source 114, which can be monitored via the battery monitor 156, can be carried in the housing 112, and can selectively operate the pump 118, and computing device 120. Control of the pump 118 can be directed by a motor drive/monitor element 158.

The computing device 120 can include a processor 140, memory 142, 144 & 146, and transceiver circuitry 148. In one embodiment, the processor 140 can be a microprocessor, logic circuit, Application-Specific Integrated Circuit (ASIC) state machine, gate array, controller, or the like. The computing device 120 can generally be configured to control infusion of infusate according to programmed parameters or a specified treatment protocol. The programmed parameters or specified treatment protocol can be stored in the memory 142, 144 & 146 for specific implementation by a control register 154. A clock/calendar element 152 can maintain system timing for the computing device 120. In one embodiment, an alarm drive 150 can be configured to activate one or more notification, alert or alarm features, such as an illuminated, auditory or vibratory alarm 160. In some embodiments, the processor 140 can be configured to selectively activate the needle detection sensor 128 and access port marker 138, prior to a physical attempt to insert a needle of the refill device into the access port 122 of the implantable device 102.

The transceiver circuitry 148 can be configured to receive information from and transmit information to the one or more physiological sensors 110, external programmer 106, and server 108. The implantable device 102 can be configured to receive programmed parameters and other updates from the external programmer 106, which can communicate with the implantable device 102 through well-known techniques such as wireless telemetry, Bluetooth, or one or more proprietary communication schemes (e.g., Tel-M, Tel-C, etc.). In some embodiments, the external programmer 106 can be configured for exclusive communication with one or more implantable devices 102. In other embodiments, the external programmer 106 can be any computing platform, such as a mobile phone, tablet or personal computer. In some embodiments, the implantable device 102 and external programmer 106 can further be in communication with a cloud-based server 108. The server 108 can be configured to receive, store and transmit information, such as program parameters, treatment protocols, drug libraries, and patient information, as well as to receive and store data recorded by the implantable device 102.

In embodiments, estimation of a volume of remaining medicament within a fluid reservoir of an implantable medical device 102 can be performed, at least partially by the programmer 106. In one embodiment, the programmer 106 or components thereof can comprise or include various modules or engines, each of which is constructed, programmed, configured, or otherwise adapted to autonomously carry out a function or set of functions. The term “engine” as used herein is defined as a real-world device, component, or arrangement of components implemented using hardware, such as by an application specific integrated circuit (ASIC) or field programmable gate array (FPGA), for example, or as a combination of hardware and software, such as by a microprocessor system and a set of program instructions that adapt the engine to implement the particular functionality, which (while being executed) transform the microprocessor system into a special-purpose device. An engine can also be implemented as a combination of the two, with certain functions facilitated by hardware alone, and other functions facilitated by a combination of hardware and software. In certain implementations, at least a portion, and in some cases all, of an engine can be executed on the processor(s) of one or more computing platforms that are made up of hardware (e.g., one or more processors, data storage devices such as memory or drive storage, input/output facilities such as network interface devices, video devices, keyboard, mouse or touchscreen devices, etc.) that execute an operating system, system programs, and application programs, while also implementing the engine using multitasking, multithreading, distributed (e.g., cluster, peer-peer, cloud, etc.) processing where appropriate, or other such techniques. Accordingly, each engine can be realized in a variety of physically realizable configurations and should generally not be limited to any particular implementation exemplified herein, unless such limitations are expressly called out. In addition, an engine can itself be composed of more than one sub-engines, each of which can be regarded as an engine in its own right. Moreover, in the embodiments described herein, each of the various engines corresponds to a defined autonomous functionality; however, it should be understood that in other contemplated embodiments, each functionality can be distributed to more than one engine. Likewise, in other contemplated embodiments, multiple defined functionalities may be implemented by a single engine that performs those multiple functions, possibly alongside other functions, or distributed differently among a set of engines than specifically illustrated in the examples herein.

In some embodiments, the programmer 106 can include a processor 162, memory 164, a control engine 166, a communications engine 168, and a power source 170. Processor 162 can include fixed function circuitry and/or programmable processing circuitry. Processor 162 can include any one or more of a microprocessor, a controller, a DSP, an ASIC, an FPGA, or equivalent discrete or analog logic circuitry. In some examples, the processor 162 can include multiple components, such as any combination of one or more microprocessors, one or more controllers, one or more DSPs, one or more ASICs, or one or more FPGAs, as well as other discrete or integrated logic circuitry. The functions attributed to processor 162 herein may be embodied as software, firmware, hardware or any combination thereof.

The memory 164 can include computer-readable instructions that, when executed by processor 162 cause ECU 120 to perform various functions. Memory 164 can include can volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. Control engine 166 can include instructions to control the components of the programmer 106 and instructions to selectively control the implantable medical device 102.

The communications engine 168 can include any suitable hardware, firmware, software, or any combination thereof for communicating with other components of the vehicle and/or external devices. Under the control of processor 162, the communication engine 168 can receive downlink telemetry from, as well as send uplink telemetry to one or more external devices (e.g., the implantable medical device 102, etc.) using an internal or external antenna. In addition, communication engine 168 can facilitate communication with a networked computing device and/or a computer network 108. For example, communications engine 168 can receive updates to instructions for control engine 166 from one or more external devices. In another example, communications engine 168 can transmit data regarding the state of system 100 to one or more one or more external devices.

Power source 170 is configured to deliver operating power to the components of the programmer 106. Power source 170 can include a battery and a power generation circuit to produce the operating power. In some examples, the battery is rechargeable to allow extended operation. Power source 170 can include any one or more of a plurality of different battery types. In some embodiments, the programmer 106 can further include an external power supply port.

In some embodiments, the medical system 100 can include a feature enabling patients to adjust administration of their respective dosage of the infusate (e.g., via the external programmer 106), within prescribed limits. For example, in some embodiments, a patient controlled activation (PCA) feature can be enabled, thereby enabling the patient to selectively administer infusate upon demand.

In conventional embodiments, where the PCA or other patient control features are enabled, an average daily remaining volume of infusate within the fluid reservoir 116 can be based on a maximum allowable activation of the PCA, such that the next refill interval would be scheduled prior to an exhaustion of the infusate—typically with a limited volume of infusate being held in reserve to ensure that the infusate is not completely exhausted prior to the next refill. Thus, such infusate estimations can be based on a “worst-case” scenario, where the medical system 100 assumes that the patient activations occur at the maximum prescribed limit. In reality, where patient controls are activated, actual dispensation of infusate is often much less than the maximum prescribed limit.

Accordingly, although complete exhaustion of the medicament reservoir can be avoided by assuming a worst-case scenario, the estimated average daily remaining volume and corresponding refill interval are often inaccurate, potentially resulting in volume discrepancy and otherwise avoidable cost related to waste of a remaining infusate (which is sometimes aspirated) during refill appointments. For example, such inaccuracies are sometimes noted by clinicians during a refill procedure, for example where the clinician withdraws the remaining infusate out of the device 102 prior to refilling the fluid reservoir 116. A comparison of the actual residual volume (ARV) to the expected residual volume (ERV) can provide insight as to the possibility of systematic under-infusion or over-infusion by the device 102.

Moreover, in addition to incorrect assumptions about a fixed average daily dose and average daily volume (e.g., particularly where the PCA or other patient control features are enabled), a number of other variables including drug type, concentration, infusion rate, off label usage, tolerances, etc. may have an effect on the ARV.

Embodiments of the present disclosure address deficiencies of past embodiments through an enhanced design by incorporating actual daily use condition data to provide a more accurate and individual patient tailored estimated average daily volume and corresponding refill interval. For example, in some embodiments, the system 100 uses a variety of statistical tools and operations to analyze historical use data to forecast a next refill interval as a random variable. In embodiments, such estimations can provide more realistic refill intervals than the past worst-case scenario estimations, as some patients may tend to administer infusate at or near the maximum prescribed limit, while other patients may administer infusate at much lower levels.

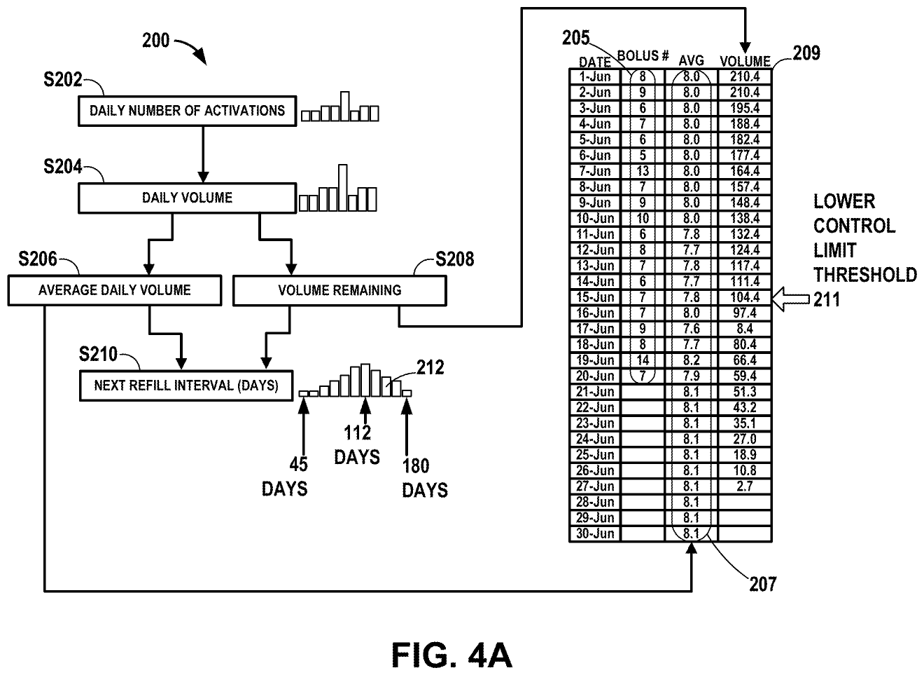

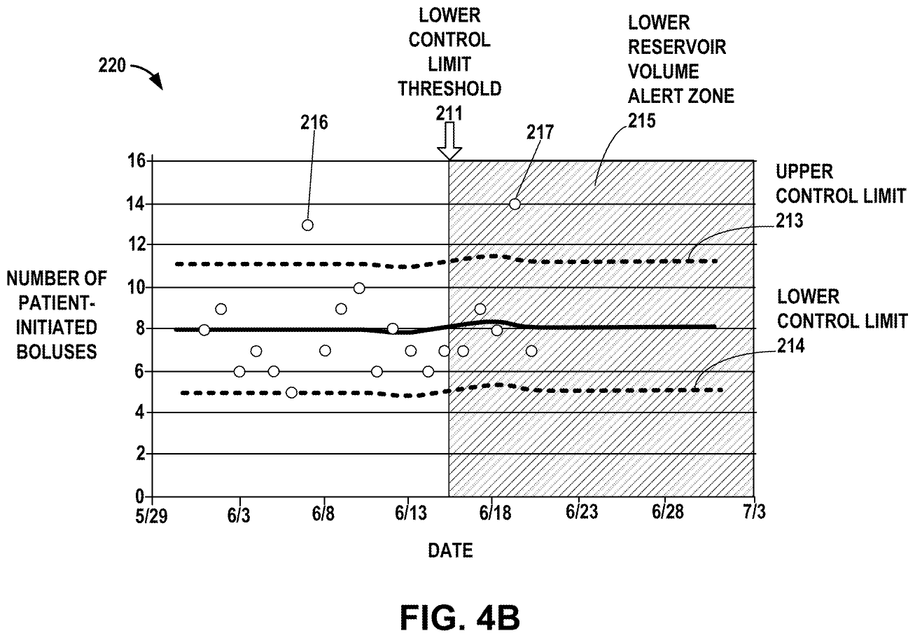

With reference to FIG. 4A, a flowchart depicting a model 200 of estimating a refill interval based on actual daily use condition data is depicted in accordance with an embodiment of the disclosure. At S202, daily patient activations of the PCA and other administrations of medicament can be recorded (e.g., via pump 102, external programmer 106, or a combination thereof). At S204, a daily volume based on the recorded daily patient activations and other scheduled infusions can be used to establish patient specific historical use condition data 205.

At S206, the patient specific historical use condition data can be extended over a period of time (e.g., via an extension of the moving average of the historical data, trend line, or the like) to establish an estimated average daily volume of administered infusate 207. As additional data points are added to the patient specific historical use condition data, the projected estimate of the average daily volume can be continually refined.

Additionally at S208, an estimated volume remaining of the infusate 209 can be calculated. For example, in some embodiments, the estimated volume can be computed by subtracting the estimated average daily volume from a prior estimated volume remaining to determine a subsequent volume remaining. At S210, a next refill interval can be computed. For example, in one embodiment, the date of a next refill interval can be established when the estimated volume remaining decreases below a defined threshold or lower control limit 211.

In some embodiments, one or more other factors with the potential to impact the actual dose or volume of infusate delivered can be added as random variables to increase a statistical fidelity of the model 200. For example, in some embodiments, a random probability distribution model (e.g., a stochastic model, Markov chain, Monte Carlo simulation etc.) can be used to estimate the average daily volume, such that the next refill interval can be presented as a distribution of probable refill dates 212, rather than a fixed estimation of the refill date.

For example, in one embodiment, a model of expected boluses (e.g., occurring on dates in the future) can be based on a normal cumulative distribution curve supplied with a random variable, given a distribution mean corresponding to an average and standard deviation of the patient specific historical use condition data 205. To compute a distribution of probable refill dates 212, the model of expected boluses can be run over a large number of iterations (e.g., 1000 iterations), with an expected refill date calculated for each iteration. A mean, median, standard deviation, and percentiles can then be calculated for the expected refill dates calculated for each iteration. The percentiles can then be used to inform a user of an estimated probability that the calculated refill date will be after a specific date, based on current use patterns (e.g., the patient specific historical use condition data 205). For example, the computed percentiles can be used to determine that there is a 75% probability that the refill date will be after June 19^{th}, and a 95% probability that the refill date will be after June 14^{th}. Other random probability distribution models are also contemplated.

Further, in some embodiments, the system 100 can employ one or more PCA controls to inhibit premature exhaustion of infusate as a result of a dramatic behavioral change prior to a scheduled refill procedure. In one embodiment, the number of patient initiated boluses or otherwise delivered infusate volume can be limited to align with the recorded patient specific historical use condition data, particularly when the reservoir volume reaches a threshold limit or low reservoir volume alert zone to inhibit inaccuracies in the forecast refill interval. For example, if in the past, the patient has always activated the PCA well below the prescribed limit, then suddenly changes behavior to dramatically increase the number of activations, a statistical algorithm can be used to detect the changing trend or use condition as an outlier. If the use condition outlier occurs within a defined low reservoir volume alert zone, one or more constraints can be applied (e.g., to limit the number of permitted boluses) to mitigate the risk of premature infusate depletion and the risk of an earlier than expected empty reservoir state.

With additional reference to FIG. 4B, a graphical depiction 220 of a daily number of patient initiated boluses with established upper control limit 213 and lower control limit 214 to inhibit premature exhaustion of infusate as a result of dramatic behavioral change prior to the scheduled refill procedure, is depicted in accordance with an embodiment of the disclosure. In some embodiments, the upper and lower control limits 213, 214 can be established based at least in part based on the recorded patient specific historical use condition data. A low reservoir volume alert zone 215 can be established based on an estimated volume remaining/refill interval.

In the example shown in FIG. 4B, two of the data points 216, 217 included in the graphical depiction represent “outliers,” in that these data points 216, 217 fall outside of the established upper and lower control limits 213, 214. In this particular example, no constraints may be applied to the first outlier 216, as although the first outlier 216 is above the established upper control limit 213, the first outlier 216 is not within the low reservoir volume alert zone 215. By contrast, one or more constraints may be applied to the second outlier 217, as the second outlier 217 is both above the established upper control limit 213 and is within the low reservoir volume alert zone 215. Accordingly, in some embodiments, actual patient activations representing point 217 may be limited to the number of patient initiated boluses represented by the upper control limit 213. Other types of limits, zones and constraints are also contemplated.

According to some embodiments, the medical system 100 can use a variety of statistical models (e.g., linear regression, Bayes theorem, etc.) as an aid in estimating a reservoir volume of the implantable device 102. For example, Bayes theorem can be useful in identifying a conditional probability associated with an estimated volume (e.g., the probability that a discrepancy between the ARV and ERV is below a threshold), given that some other event has already occurred or is presently occurring.

For illustration, a user may speculate that use of a particular type of drug or infusate is more likely to lead to a discrepancy between the ARV and ERV. Bayes theorem can be used to quantify this premise or speculation, by identifying the conditional probability of there being a discrepancy between the ARV and ERV exceeding a defined threshold (e.g., 5%), if a particular type of drug is being used in the pump. In computing the conditional probability, a record of past events or transactions (sometimes referred to as “control data”) can be analyzed. The control data can be specific to one user, or can be gathered from a larger population of users for later application to a specific user. For example, the control data can be gathered in part from other implantable pumps 102 in the same family to reduce uncertainty.

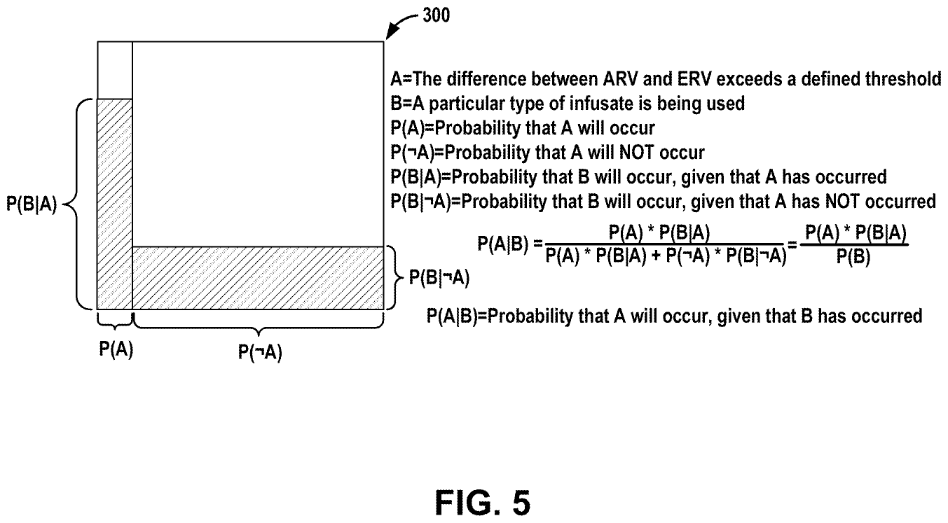

With reference to FIG. 5, the control data 300 can be used to determine what portion of the data resulted in a discrepancy between the ARV and ERV exceeding a defined threshold, regardless of what type of infusate is being used. In this particular illustration, data representing a sampling of 1000 pumps is used. The total control data 300 sampling can be divided into two portions, represented as P(A), the probability that the discrepancy between the ARV and ERV exceeds a defined threshold, before considering other events (sometimes referred to as the “prior”), and P(¬A), the probability that the discrepancy between the ARV and ERV does not exceed a defined threshold, before considering other events.

In this illustration, it is found that 50 out of 1000 data points, the discrepancy between the ARV and ERV exceeds a defined threshold (i.e., P(A)=0.05), before other evidence was considered. In other words, regardless of what type of infusate is used, 5% of the time the estimated volume was determined to be inaccurate (e.g., at the time of a refill procedure). Conversely, again without considering the type of infusate being used, 950 out of 1000 data points resulted in the ERV being within a defined threshold of the ARV (i.e., P(¬A)=0.95).

Additionally, the control data is examined to determine the probability of finding that the discrepancy between the ARV and ERV exceeds a defined threshold when it is known that a particular type of drug (e.g., Bupivacaine, Fentanyl, etc.) is being used, which can be represented as P(B|A) (sometimes referred to as the “likelihood”). The data is also examined to determine P(B|¬A), representing times a probability of the ERV being within a defined threshold of the ARV, even though a particular type of drug is being used. In this illustration, it is found that in 42 of the 50 times where a discrepancy between the ARV and ERV was found, the particular type of drug had been used (i.e., P(B|A)=0.84). Conversely, it was found that in 228 of the 950 times that the ERV was within a defined threshold of the ARV, the particular type of drug was also being used (i.e., P(B|¬A)=0.24) (sometimes referred to as a “false positive”).

With this data, it is possible to infer a probability of there being a discrepancy between the ARV and ERV exceeding a defined threshold, given that a particular type of infusate is being used according to Bayes theorem. The formula for Bayes theorem follows:

$$P ( A \vert B ) = \frac{P ( A ) \cdot P ( B \vert A )}{P ( A ) \cdot P ( B \vert A ) + P ( ¬ A ) \cdot P ( B \vert ¬ A )} = \frac{P ( A ) \cdot P ( B \vert A )}{P ( B )}$$

Accordingly, based on the illustration, P(A|B) is about 0.156, meaning that if the medical system 100 begins with the knowledge that a particular infusate known to result in larger discrepancies between ARV and ERV is being used, there will be a 15.6% probability that the discrepancy exceeds the defined threshold. Comparing P(A|B) with P(A) (representing discrepancies exceeding a defined threshold without knowledge of the infusate being used) shows that knowing what type of infusate is being used increases the likelihood of prediction by a factor of three.

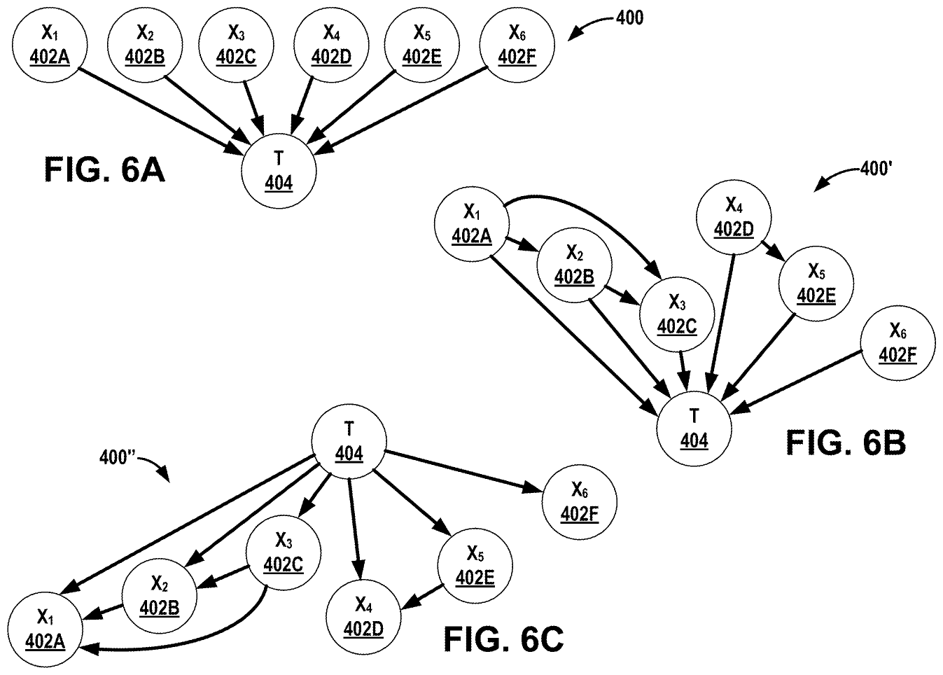

With reference to FIGS. 6A-C, in more complex models, Bayes theorem can be used to predict the probability of a target (T) occurring (e.g., the probability of a discrepancy between the ARV and ERV exceeding a defined threshold) based on a plurality of causal factors or variables, sometimes referred to as predictors or factors (Xₙ). Accordingly, in some embodiments, the predictors can be linked to the target, and potentially to each other, to form a Bayesian network. Like the previous example, in some embodiments, the Bayesian network can represent a form of supervised learning, in which the links between the predictors and the target, as well as the initial probabilities are established based on control data 300 (e.g., gathered in part from other implantable pumps 102 in the same family). In such cases, the control data 300 can be used to map the predictors Xₙ to the target.

In the simplest case, all of the predictors can be independent from one another, in that a combination of any two predictors does not increase the probability of the target occurring. This type of network 400 (as depicted in FIG. 6A) is sometimes referred to as a naïve Bayesian network. In more complex systems, there may be a connection between predictors (Xₙ) 402A-F. For example, it may be found that the probability of the target (T) 404 occurring is relatively low when either X₁ or X₂ occurs independently, but that the probability of the target occurring jumps unexpectedly when both X₁ and X₂ occur. In this case, a link may be established between X₁ and X₂. This type of network 400′ (as depicted in FIG. 6B) is sometimes referred to as an augmented naïve Bayesian network.

As the links between the predictors are initially unknown, one problem in establishing a supervised learning network is the requirement for extremely large sets of control data. Specifically, in order to establish the links between the nodes of the network, a conditional probability distribution of T given every combination of the predictors (Xₙ) is required. For example, if there are ten predictors, more than three million probability distributions would be required to establish the causal structure. Instead, a procedure is often taken to invert the causal structure 400″ (as depicted in FIG. 6C), at least initially, by making 404 (T) the parent of the 402A-F (Xₙ). Even though inverting the causal structure violates the independence assumption, the inversion still often obtains good prediction performance.

In the example inverted augmented naïve Bayesian network of FIG. 6C, the conditional probability of the target occurring can be computed according to the following formula:

P(T|Data)=K·P(X₃|X₁,X₂,T)·P(X₂|X₁,T)·P(X₁|T)·P(X₅|X₄,T)·P(X₄|T)·P(X₆|T)·P(T)

where K equals a normalizing constant. Accordingly, to establish Bayesian network 400 from control data on a set of individuals, the conditional probability distribution of each predictor (at least initially considered as an effect) is computed, given that the target has occurred. Once the Bayesian network is established, it is possible to develop inferences using new data (e.g., data on particular potential customer) to determine the likelihood that T will occur based on a plurality of predictors Xₙ, such as drug type, concentration, infusion rate, off-label usage, manufacturing tolerances, and the like.

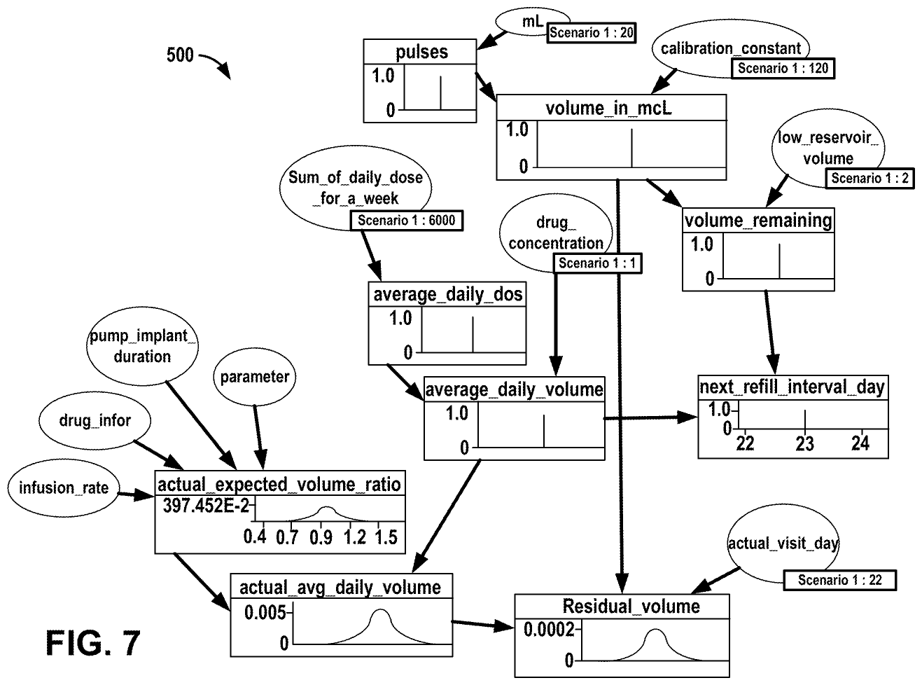

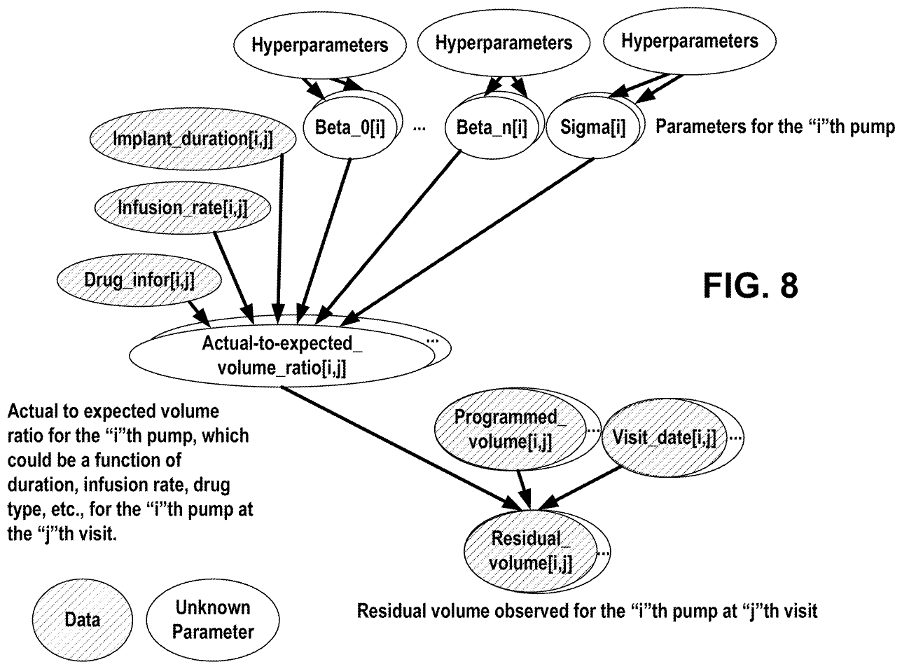

With additional reference to FIGS. 7 & 8, in some embodiments, the network model 500/500′ can be used to estimate the actual daily volume a given device 102. As depicted, the model can be used to link parameters throughout the model 500. In some embodiments, the parameters can be modeled as random draws from distributions dependent upon hyper parameters, such that the parameters can be estimated as a weighted average of a collection of pooled control data 300 as well as unique observations from a particular device 102. Accordingly, the model 500/500′ enables capturing of trends in the population, as well as unique characteristics of individual devices 102. In addition to the variables discussed above, data collected during refill procedures (e.g., ARV, implant duration, drug type, concentration, infusion rate, etc.) can be used to update the model 500/500′. Further, predictors or factors can be learned from the control data 300, according to any of the various methods described above.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A medical system configured to estimate a volume of infusate within an implantable medical device, the system comprising:
an implantable medical device comprising a fluid reservoir; and
an external programmer in communication with the implantable medical device, the external programmer comprising a processor configured to estimate a distribution of refill intervals based on actual infusion data including patient initiated infusions.

2. The system of claim 1, wherein the distribution of refill intervals is determined by a random probability distribution model.

3. The system of claim 1, wherein the random probability distribution model is at least one of a stochastic model, Markov chain or Monte Carlo simulation.

4. The system of claim 1, wherein the distribution of refill intervals is used to determine a scheduled refill date based on a probability that the scheduled refill date will occur after a termination date of a majority of the distribution of refill intervals.

5. The system of claim 4, wherein at least 95% of the distribution of refill intervals end before the scheduled refill date.

6. The system of claim 1, wherein the system implements one or more controls to inhibit premature exhaustion of infusate, when an estimated fluid reservoir volume decreases below a threshold limit.

7. The system of claim 6, wherein the actual infusion data including patient initiated infusions is used to establish an upper control limit.

8. The system of claim 7, wherein patient initiated infusions are limited when the actual infusion data including patient initiated infusions at least one of approaches or is greater than or equal to the upper control limit.

9. The system of claim 1, wherein at least one condition having a potential to impact an actual residual volume of infusate delivered by the implantable medical device is considered in the estimation of the distribution of refill intervals.

10. The system of claim 9, wherein the at least one condition includes a measured actual residual volume, drug type, off-label usage, infusion rate, concentration, implant duration, manufacturing tolerance, or combination thereof.

11. The system of claim 1, wherein the processor is further configured to estimate a probability of an expected residual volume of infusate within the fluid reservoir differing from an actual residual volume of infusate within the fluid reservoir by greater than or equal to a determined percentage, based on an occurrence of at least one condition.

12. The system of claim 11, wherein the probability of an expected residual volume differing from an actual residual volume is determined by at least one of a Baysian network or other statistical model.

13. A medical system configured to estimate a volume of infusate within an implantable medical device, the system comprising:
an implantable medical device comprising a fluid reservoir; and
an external programmer in communication with the implantable medical device, the external programmer comprising a processor configured to estimate a probability of an expected residual volume of infusate within the fluid reservoir differing from an actual residual volume of infusate within the fluid reservoir by greater than or equal to a determined percentage, based on an occurrence of at least one condition.

14. The system of claim 13, wherein the at least one condition includes a measured actual residual volume, drug type, off-label usage, infusion rate, concentration, implant duration, manufacturing tolerance, or combination thereof.

15. The system of claim 13, wherein the probability of an expected residual volume differing from an actual residual volume is determined by a at least one of a Baysian network or other statistical model.

16. The system of claim 13, wherein the probability of an expected residual volume differing from an actual residual volume is determined as a distribution of values, each value representing a possible difference between an expected residual volume and an actual residual volume.

17. The system of claim 16, wherein the distribution of values is determined by a random probability distribution model.

18. The system of claim 17, wherein the random probability distribution model is at least one of a stochastic model, Markov chain or Monte Carlo simulation.

19. A method of estimating a volume of infusate within an implantable medical device, the method comprising:
collecting actual infusion data including patient initiated infusions from an implantable medical device including a fluid reservoir;
estimating a distribution of refill intervals based on the actual infusion data.

20. The method of claim 19, wherein the distribution of refill interval is based on controlled data gathered from a sampling of implantable medical devices.
